(19) United States
(12) Patent Application Publication
(10) Pub. No.: US 2003/0138953 A1
(43) Pub. Date: Jul. 24, 2003

(54) Method for the generation of 3' RNA fragments and N-terminally truncated polypeptides

(76) Inventors: Fritz Von Weizsacker, Freiburg (DE); Christian Thoma, Pforzheim (DE); Wolf-Bernhard Offensperger, Ehrenkirchen (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/056,161

(22) Filed: Jan. 24, 2002

(51) Int. Cl. C12N 15/74; C12N 5/00

(52) U.S. Cl. 435/375; 435/471

(57) ABSTRACT

The present invention provides a method for the generation of a 3′ RNA cleavage fragment from a target RNA using antisense oligonucleotides (AS ODNs) as well as a method for the generation of N-terminally truncated polypeptides translated from said 3′ RNA cleavage fragment. Finally, the present invention provides several uses of said methods, e.g. (i) the study of the stability and translational efficiency of uncapped mRNAs, (ii) the generation of novel polypeptides with desired properties from an endogenous cellular repertoire or (iii) the evaluation of possible side effects of an antisense therapy.

### FIELD OF THE INVENTION

The present invention relates to the use of antisense oligonucleotides (AS ODNS) for the generation of specific RNA fragments which may, preferably, serve as mRNA templates.

### BACKGROUND OF THE TECHNOLOGY

According to current concepts mRNAs can form closed loops by the interaction of their 5′ and 3′ ends with associated factors promoting initiation of translation and stability. mRNA degradation is then thought to occur after disruption of this functional messenger ribonucleoprotein (mRNP) particle. At least three types of such events have been determined: poly(A) tail shortening, arrest of translation at a premature nonsense codon (mRNA surveillance) and endonucleolytic cleavage. Steps subsequent to poly(A) tail shortening or premature translational termination lead either to the removal of the 5′ cap followed by 5′-3′ exonucleolytic digestion or to 3′-5′ decay of the mRNA body. mRNA fragments generated by endonucleolytic cleavage are most likely removed by exonucleolytic decay as well, but these events have not been characterized in detail.

An alternative, but clinically important way to induce such endonucleolytic cleavage of a target mRNA would be the application of antisense oligodeoxynucleotides (AS ODNs). AS ODNs are known to trigger pleiotropic effects such as translation arrest, intracellular signaling pathway activation and CpG-mediated B-cell stimulation. The most effective mechanism, however, is attributed to ribonuclease H (RNase H)-mediated target-mRNA degradation. This sequence-specific antisense mechanism is based on the hybridization of the oligonucleotide to a complementary region in the target message. Oligonucleotides possessing anionic internucleoside linkages, either unmodified phosphate diesters or sulfur-modified phosphorothioates, form heteroduplexes that are substrates for the ubiquitous RNase H. RNase H cleaves the mRNA component of the heteroduplex. It is generally assumed that the two resulting mRNA cleavage fragments are rapidly degraded in the cell, thus rendering the message permanently untranslatable.

It is now tempting to integrate this RNase H-mediated endonucleolysis into the above-mentioned concepts of mRNA degradation. From a theoretical point of view two fragments are resulting from this antisense-mediated endonucleolysis: a 5′ fragment possessing a cap and lacking a poly(A) tail and a 3′ fragment possessing a poly(A) tail and lacking a cap. The lack of the poly(A) tail of the 5′ fragment is thought to lead to subsequent 3′-5′ exonucleolytic decay and/or 5′-3′ exonucleolysis if decapping occurs. The integrity of the poly(A) tail of the 3′ fragment is thought to be unchanged whereas it may be a substrate for 5′-3′ exonucleolysis. There is increasing knowledge about the central role of the poly(A) tail and its associated factors in the cytoplasmic fate of a mRNA. Formation of a functional mRNP via a closed loop enables cap-dependent and cap-independent translation and protects against exonucleolytic degradation. AS ODN-mediated RNase H cleavage products have been reported in different settings, including liver specimens from rats treated with AS ODNs in vivo, Xenopus oocytes, chinese hamster ovary cells, human leukemia and H9 cells. However, the fate of these fragments in terms of RNA stability and their mechanisms of degradation are largely unknown and so far there is no method available allowing to generate stable 3′ mRNA fragments from a target mRNA in vivo.

### SUMMARY OF THE INVENTION

The present invention is based on the unexpected finding that cleavage of target mRNAs within the AS ODN binding region results in subsequent degradation of the 5′ but not the 3′ cleavage product. The full length mRNA was undetectable following AS ODN treatment. It was found that some, if not all 3′ mRNA fragments lacked a 5′ cap structure whereas their poly(A) tail length remained unchanged. They were furthermore efficiently translated into N-terminally truncated proteins as demonstrated in three settings: production of shortened hepadnaviral surface proteins, alteration of the subcellular localization of a fluorescent protein and shift of the transcription factor C/EBP_{α} isoform expression levels. Thus, AS ODN treatment may result in the synthesis of N-truncated proteins with biologically relevant effects. In conclusion, the findings of the present invention suggest a new mechanism of AS ODN action: target mRNAs are cleaved by RNase H into two fragments. The capped 5′ fragment lacking a poly(A) tail is rapidly degraded whereas the 3′ fragment appears to be stable, perhaps due to its poly(A) tail and binding factors. This fragment can then be translated into protein.

These findings have several implications for both cellular biology and drug therapy. First, a useful model for the study of stability and translational efficiency of probably uncapped mRNAs is provided. Second, the AS ODN strategy of the present invention could be used to generate novel polypeptides with desired properties from the endogenous cellular repertoire. As shown here for the transcription factor C/EBP_{α}, promising targets may be proteins that are translated into functionally different isoforms by leaky scanning such as transcription factors or protooncogenes, e.g., C/EBP_{α}, C/EBP_{β}, int-2, BAG-1. Third, the therapeutic use of AS ODN may have to be reevaluated in the light of the findings of the present invention, since the translation of novel, N-terminally truncated polypeptides with unexpected and biologically relevant effects may cause serious side effects of antisense therapy. Fourth, non-translated the strategy of the present invention may be used to generate non-translated RNA fragments which may serve as decoy and, thus, be useful for interfering with a variety of cellular functions as well as the life cycle of viruses and microorganisms.

The present invention, thus, provides a method for the generation of a stable 3′ RNA cleavage fragment by use of AS ODNs as well as a method for the generation of an N-terminally truncated polypeptid. Said method is useful for (i) studying the stability and translational efficiency of truncated mRNAs, (ii) generating novel polypeptides with desired properties from an endogenous cellular repertoire or (iii) evaluating possible side effects of an antisense therapy.

### DESCRIPTION OF THE DRAWINGS

FIG. 1: AS ODNs mediate cleavage but not complete degradation of the target mRNA

(A) The DHBV pregenomic (pg) and subgenomic (sg) mRNAs overlap as indicated and the AS ODN and probe binding sites on the mRNAs are illustrated.

(B) Northern blot analysis with oligo hybridization of viral RNA isolated from cotransfected avian hepatoma cells. The pg mRNA is no longer detectable upon cotransfection of AS ODN DHBV795 while a distinct band slightly smaller than sg mRNA was generated. Co: non transfected cells, wt: DHBV transfected cells.

(C) Similar results were observed in human HuH-7 hepatoma cells using AS ODNs DHBV795 and DHBV874, even if the AS ODN mediated cleavage by the latter AS ODN was not complete. Equal amounts of RNA were loaded.

FIG. 2: Capping status of the mRNA 3′ cleavage fragments and determination of Rnase H-mediated cleavage sites

(A) mRNA 3′ cleavage fragments were examined by ligation and subsequent RT-PCR (see Example 1 for detailed description).

(B) PCR using primers 860− and 2665+ resulted in a 0.4 kbp fragment in uncapped ligated mRNAs (lane 4). Neg co: water, pos co: DHBV expression plasmid. 1.3 and 0.8 kbp fragments were due to amplification from the pg mRNA (lanes 1,2) and/or expression plasmid (lane 7) but not the sg mRNA (lane 3) and did not necessitate the ligation step.

(C) Sequence analysis of cloned PCR products. A representative sequence gel of one clone showing the junction of the 5′ end of the cleaved 3′ fragment to the poly(A) tail is shown.

(D) Schematic presentation of the AS ODN mediated cleavage sites indicated by arrows.

FIG. 3: Determination of poly(A)tail length Poly(A) tail length of 3′ cleavage fragments was not significantly different from uncleaved DHBV RNA. RNA from DHBV cells cotransfected without (lanes 1-4) and with (lanes 5-8) AS ODN DHBV795 was hybridized in vitro to the indicated oligos and cleaved by RNase H. This reaction resulted in 3′ UTR fragments with the indicated size without (lanes 2,3,6,7) and with a poly(A) tail (lanes 4,8).

FIG. 4: Detection of N-terminally truncated preS/S proteins in AS ODN treated cells Western blot analysis of viral preS/S proteins isolated from cotransfected avian LMH (A) and human HuH-7 hepatoma cells (B). Co: non transfected, wt: DHBV transfected cells. Note the relative abundance of gp 35 and gp 33 in cells treated with AS ODN DHBV795 and the abundance of gp30 in cells treated with AS ODN DHBV874. Equal amounts of protein were loaded.

FIG. 5: AS ODN-mediated translation of truncated ECFP-Golgi proteins rearranges the subcellular fluorescence

(A) Fluorescence microscopy of cotransfected avian hepatoma cells. ECFP-Golgi fluorescence is localized only in the cytoplasm and rearranged throughout the cell after AS treatment (upper panels). The cell nuclei were stained by propidium iodide as controls (lower panels). The same time was chosen for exposures.

(B) Again, Northern blot analysis revealed the generation of stable 3′ cleavage fragments and the degradation of the 5′ fragments in avian hepatoma cells by hybridization with probes located 3′ (Probe 1) or 5′ (Probe 2) of the AS ODN binding site.

(C) Western blot analysis of Golgi-ECFP proteins isolated from cotransfected avian hepatoma cells. Co: non transfected, wt: ECFP-Golgi transfected cells. Note that upon AS treatment a smaller protein was detected in cotransfected cells. Schematic representation of the Golgi-ECFP mRNA with the initiation sites indicated in relation to the protein bands on the left. Equal amounts of RNA and protein were loaded.

FIG. 6: AS ODN shifts C/EBP_{α} isoform expression towards p30 Western blot analysis of C/EBP_{α} isoforms isolated from cotransfected avian hepatoma cells. Neg. co: non transfected, wt: C/EBP_{α} transfected cells. Note the relative abundance of p30 in treated cells. Equal amounts of protein were loaded.

### DETAILED DESCRIPTION OF THE INVENTION

The present invention relates to a method for the generation of a 3′ RNA cleavage fragment from a target RNA, said method comprising the following steps:

(a) transfecting a cell containing a target RNA or an extract of said cell with an antisense oligonucleotide capable of hybridizing to said target RNA or an expression vector containing a DNA molecule which is operatively linked to regulatory elements allowing transcription of said antisense oligonucleotide in said cell; and

(b) maintaining or culturing said cell or incubating said extract for a sufficient period of time and under conditions such that said target RNA is degraded and a stable 3′ RNA cleavage fragment produced.

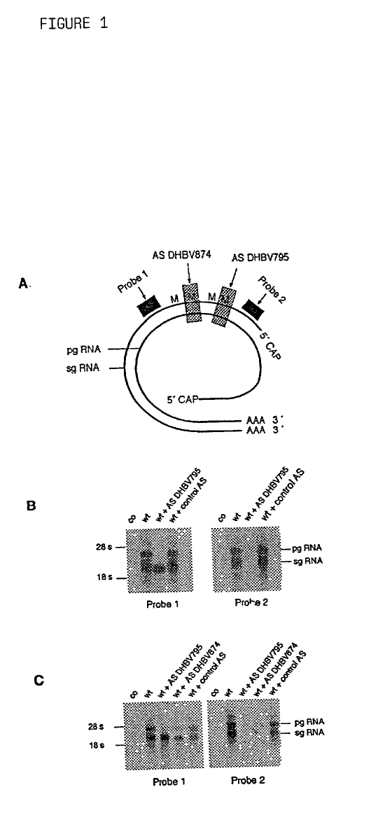

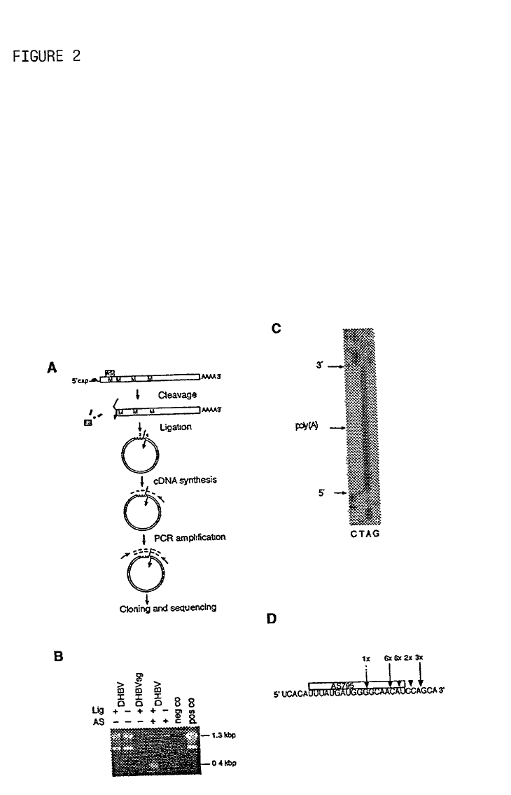

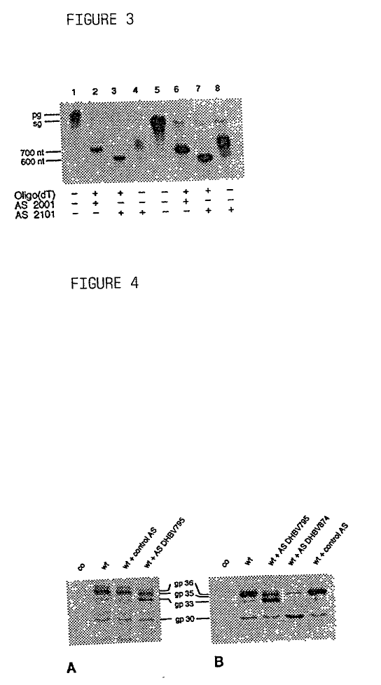

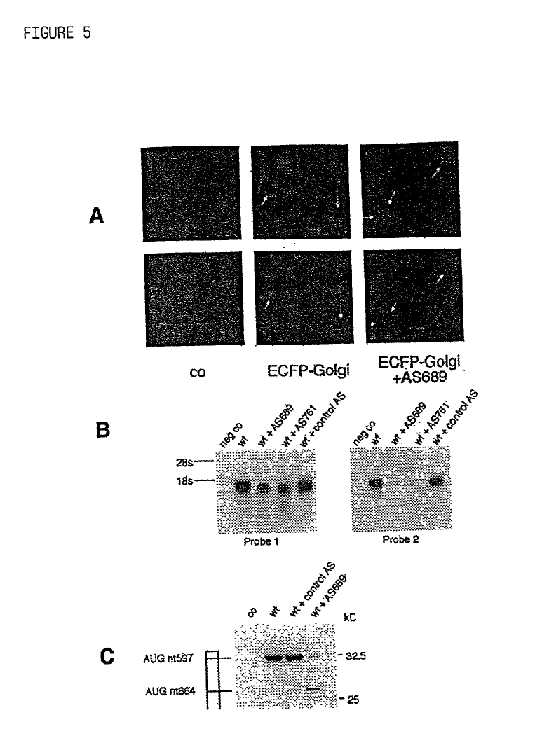

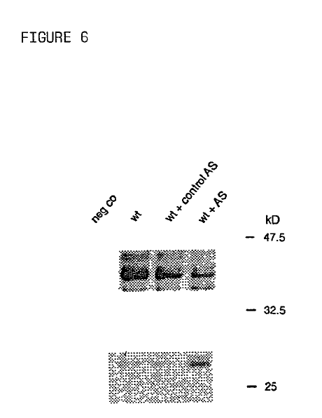

As used herein, the term “3′ RNA cleavage fragment” relates to a fragment located downstream of the hybridization site of the antisense oligonucleotide (AS ODN); see FIG. 1D. The 5′ end of said 3′ RNA cleavage fragment is, preferably, located 0 to 8 nucleotides upstream or downstream of the nucleotide of the target RNA hybridizing to the nucleotide of the 5′ end of the AS ODN.

As used herein, the term “target RNA” comprises any RNA and is not restricted to an RNA naturally occuring in a host cell. Preferably said target RNA is an mRNA.

As used herein, the term “extract” comprises disrupted and/or lysed cells or a cell-free extract which still allows RNase H-mediated endonucleolysis of the AS ODN/target RNA-hybrid and, preferably, translation. Examples of such extracts include cell-free transcription-translation systems (e.g. wheat germ or rabbit reticulocyte lysate).

As used herein, the term “cell” comprises any cell. Said cell can be part of a tissue or an organism, preferably a mammal.

The antisense oligonucleotides of the present invention can be both DNA and RNA molecules, preferably ribozymes, e.g. synthetic ribozymes, with oligodeoxynucleotides being preferred. Ribozymes which are composed of a single RNA chain are RNA enzymes, i.e. catalytic RNAs, which can intermolecularly cleave a target RNA, for example an mRNA. It is now possible to construct ribozymes which are able to cleave the target RNA at a specific site by following the strategies described in the literature. (see, e.g., Tanner et al., in: Antisense Research and Applications, CRC Press Inc. (1993), 415-426). The two main requirements for such ribozymes are the catalytic domain and regions which are complementary to the target RNA and which allow them to bind to its substrate, which is a prerequisite for cleavage.

The antisense oligonucleotides of the present invention can be synthesized according to known methods or can be the products of in vitro transcription. Direct chemical synthesis can be accomplished by methods such as the phosphotriester method of Narang et al., 1979, Meth. Enzymol. 68:90; the phosphodiester method of Brown et al., Meth. Enzymol. 68:109 (1979); the diethylphosphoramidite method of Beaucage et al., Tetra. Lett., 22:1859 (1981); and the solid support method of U.S. Pat. No. 4,458,066.

Generally, to assure specific hybridization, the sequence of the antisense oligonucleotide is substantially complementary to the target RNA sequence. In certain embodiments, the antisense sequence is exactly complementary to the target sequence. The antisense oligonucleotides may also include, however, nucleotide substitutions, additions, deletions, transitions, transpositions, or modifications as long as specific binding to the relevant target sequence of the RNA is retained as a functional property of the oligonucleotide.

The person skilled in the art can easily determine whether the cell containing the target RNA and the AS ODN has been cultured for a sufficient period of time or whether the extract containing the target RNA and the AS ODN has been incubated for a sufficient period of time according to conventional methods (see, e.g., Sambrook et al., 1989, Molecular Cloning, A Laboratory Manual, 2^{nd} edition Cold Spring Harbor Laboratory Press, Cold Spring Harbor, N.Y.)., e.g. by collecting samples and determining whether the target RNA has been sufficiently degraded, e.g. by determining the 5′ end of the 3′ RNA cleavage products by suitable methods. Suitable detection methods include Northern blot analysis, RNase protection, in situ methods, e.g. in situ hybridization, in vitro amplification methods (PCR, RT-PCR, LCR, QRNA replicase or RNA-transcription/amplification (TAS, 3SR), reverse dot blot disclosed in EP-B1 0 237 362)).

A preferred embodiment of the method of the present invention additionally comprises the step of detecting the 3′ RNA cleavage fragment in the cell or extract. Methods for detecting the 3′ RNA cleavage fragment in the host cell or the extract are known to the person skilled in the art.

Preferably, the antisense oligonucleotides useful for the method of the present invention have a length of at least 8, preferably at least 18 and, more preferably, at least 25 nucleotides.

It will be appreciated that the antisense oligonucleotides of the invention can be made using nonstandard bases (e.g., other than adenine, cytidine, guanine, thymine, and uridine) or nonstandard backbone structures to provide desirable properties (e.g., increased nuclease-resistance, tighter-binding, stability or a desired T_{M}). Techniques for rendering oligonucleotides nuclease-resistant include those described in PCT publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates. Still other useful antisense oligonucleotides may contain alkyl and halogen-substituted sugar moieties comprising one of the following at the 2′ position: OH, SH, SCH₃, F, OCN, OCH₃OCH₃, OCH₃O(CH₂)ₙCH₃, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl etc. Other embodiments may include at least one modified base form or “universal base” such as inosine, or inclusion of other nonstandard bases such as queosine and wybutosine as well as acetyl-, methyl-, thio- and similarly modified forms of adenine, cytidine, guanine, thymine, and uridine which are not as easily recognized by endogenous endonucleases. Also useful are oligonucleotides having backbone analogues such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, chiral-methyl phosphonates, nucleotides with short chain alkyl or cycloalkyl intersugar linkages, short chain heteroatomic or heterocyclic intersugar (“backbone”) linkages, or CH₂—NH—O—CH₂, CH₂—N(CH₃)—OCH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂), or mixtures of the same. Also useful are antisense oligonucleotides having morpholino backbone structures (U.S. Pat. No. 5,034,506). Useful references include Oligonucleotides and Analogues, A Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan et al., Jul. 9, 1993, J. Med. Chem. 36(14):1923-1937; Antisense Research and Applications (1993, CRC Press), in its entirety and specifically Chapter 15, by Sanghvi, entitled “Heterocyclic base modifications in nucleic acids and their applications in antisense oligonucleotides.” Antisense Therapeutics, ed. Sudhir Agrawal (Humana Press, Totowa, N.J., 1996).

Furthermore, the person skilled in the art is well aware that it is also possible to label the antisense oligonucleotide with an appropriate marker for specific applications. A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include radionuclides, enzymes, fluorescent, chemiluminescent, or chromogenic agents as well as substrates, cofactors, inhibitors, magnetic particles and the like. Patents teaching the use of such labels include U.S. Patents U.S. Pat. No. 3,817,837; U.S. Pat. No. 3,850,752; U.S. Pat. No. 3,939,350; U.S. Pat. No. 3,996,345; U.S. Pat. No. 4,227,437; U.S. Pat. No. 4,275,149 and U.S. Pat. No. 4,366,241.

In a further preferred embodiment of the method of the present invention the antisense oligonucleotide is phosphorotioate-modified. Such oligonucleotides can be synthesized according to well known methods, e.g. the methods described in the literature cited above.

In a further preferred embodiment of the method of the present invention, the 3′ RNA cleavage fragment is produced by transfecting a cell with an expression vector containing a DNA sequence the transcription of which leads to the synthesis of the antisense oligonucleotide as antisense RNA or as a ribozyme. Suitable expression vectors for the transcription of antisense oligonucleotides or ribozymes are based on plasmids, cosmids, viruses, bacteriophages and other vectors usually used in the field of genetic engineering. Vectors suitable for use in the present invention include, but are not limited to the T7-based expression vector for expression in bacteria, the pMSXND expression vector for expression in mammalian cells and baculovirus-derived vectors for expression in insect cells. Preferably, the DNA molecule which can be transcribed into an antisense oligonucleotide or ribozyme is operatively linked to the regulatory elements in the recombinant vector of the invention that guarantee the transcription and synthesis of an RNA in prokaryotic and/or eukaryotic cells. The nucleotide sequence to be transcribed can be operably linked to a promoter like a T7 promoter, polyhedrin promoter, the lac promoter, the hybrid lacZ promoter of the Bluescript7 phagemid [Stratagene, La Jolla, Calif.] or pSport1 [Gibco BRL], phage lambda promoter systems, a tryptophan (trp) promoter system, a metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter etc. The expression vectors can be transferred into the chosen cell by well-known methods such as calcium chloride transformation (for bacterial systems), electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223), agent-enhanced uptake of DNA, and ex vivo transduction.

Any cell or any extract prepared from such a cell is suitable for the method of the present invention. A suitable cell is understood to be capable to take up in vitro recombinant DNA and, if the case may be, to synthesize the polypeptids encoded from the 3′ RNA cleavage fragments. Preferably, these cells are prokaryotic or eukaryotic cells, for example mammalian cells, bacterial cells, insect cells or yeast cells. Cell extracts having the biological activities described above can be prepared according to standard procedures, e.g. methods described in Sambrook et al., 1989, Molecular Cloning, A Laboratory Manual, 2^{nd} edition Cold Spring Harbor Laboratory Press, Cold Spring Harbor, N.Y.).

In an alternative embodiment, the present invention relates to a method for the preparation of an N-terminally truncated polypeptid, said method comprising the following steps:

(a) transfecting a cell containing a target mRNA or incubating an extract obtained from said cell with an antisense oligonucleotide capable of hybridizing to said target mRNA or an expression vector containing a DNA molecule which is operatively linked to regulatory elements allowing transcription of said antisense oligonucleotide in said cell;

(b) maintaining or culturing said cell or incubating said extract for a sufficient period of time and under conditions such that said target mRNA is degraded and a polypeptid translated from the 3′ mRNA cleavage product; and, optionally,

(c) detecting and functionally characterizing an N-terminally truncated polypeptid translated from a 3′ mRNA cleavage product as a result of steps (a) and (b).

The polypeptide translated from the 3′ mRNA cleavage product can be assayed according to well known methods including immunoassays, such as Western blot analysis, the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

If desired, recovery and purification of the desired polypeptides from the cell or extract may be carried out by conventional means including preparative chromatography and immunological separations involving affinity chromatography with monoclonal or polyclonal antibodies. The term “antibody” as used herein, preferably, relates to antibodies which consist essentially of pooled monoclonal antibodies with different epitopic specificities, as well as distinct monoclonal antibody preparations. Monoclonal antibodies are made from an antigen containing a fragment of the N-terminally truncated polypeptide by methods well known to those skilled in the art (see, e.g., Köhler et al., Nature 256 (1975), 495). As used herein, the term “antibody” (Ab) or “monoclonal antibody” (Mab) is meant to include intact molecules as well as antibody fragments (such as, for example, Fab and F(ab′)2 fragments) which are capable of specifically binding to the protein. Routine protein purification methods, such as ammonium sulfate precipitation, size-exclusion, anion and cation exchange chromatography, gel electrophoresis and the like (see, generally, R. Scopes, Protein Purification, Springer-Verlag, N.Y. (1982) and Deutscher, Methods in Enzymology Vol. 182: Guide to Protein Purification, Academic Press, Inc. N.Y. (1990)) can also be used instead of, or in addition to, immunoaffinity methods.

Finally, the present invention provides several uses of the methods described above, e.g. studying the stability and translational efficiency of uncapped nRNAs, generating novel polypeptides with desired properties from an endogenous cellular repertoire, evaluating possible side effects of an antisense therapy or generating non-translated RNA fragments which may serve as decoy and, thus, be useful for interfering with a variety of cellular functions as well as the life cycle of viruses and microorganisms.

### EXAMPLES

The following Examples are intended to illustrate, but not to limit the invention. While such Examples are typical of those that might be used, other methods known to those skilled in the art may alternatively be utilized.

### Example 1

Materials and Methods

(A) Plasmids

Wild-type DHBV surface proteins were expressed from plasmid pGemDHBV, a replication-competent head-to-tail dimer construct (von Weizsäcker et al., J.Virol.69 (1995), 2704-2707). pDHBVsg is a plasmid encoding only the DHBV sg RNA and was obtained by digestion of pGemDHBV with DraI and DraIII and insertion of a resulting 2060 bp fragment into the pCDNA3.1 vector (Invitrogen, Groningen, Netherlands). pECFP-Golgi (Clontech, Heidelberg, Germany, Cat. #6908-1) encodes a fusion protein consisting of enhanced cyan fluorescent protein and a sequence encoding the N-terminal 81 amino acids of human beta 1.4-galactosyltransferase. pCMVex m-alpha encodes the transcription factor C/EBP_{α} driven by a CMV promotor.

(B) Antisense Oligonucleotides

Phosphorothioate-modified, HPLC-purified ODNs were obtained from Microsynth (Balgach, Switzerland). AS ODN DHBV795: ATGTTGCCCCATCATAAA; AS ODN DHBV874: TTGGGATCATTCTTCCGG; AS ODN ECFP-Golgi689: GTGACGCCAAGGTGCAGA; AS ODN ECFP-Golgi761: TGCAGCGGTGTGGAGACT; AS ODN C/EBP_{α}516: GGAGTCGGCCGACTCCAT.

(C) Cell Culture and Transfections

LMH chicken hepatocellular carcinoma cells (Kawaguchi et al., Cancer Res.47 (1987), 4460-4463) were cultured in Isocove's modified Dulbecco's medium (GibcoBRL, Life Technologies, Karlsruhe, Germany). Human hepatoma cell lines HepG2 and HuH-7 were cultured in DMEM (PAN Biotech, Aidenbach, Germany). Media contained 8% fetal bovine serum (Biochrom, Berlin, Germany), Penicillin (100 U/ml), Streptomycin (100 μg/ml, both GibcoBRL) and 1% of a nonessential amino acid stock solution (GibcoBRL). Cells were transfected by the calcium phosphate method. In a typical cotransfection experiment, 5 μg of plasmid and 5 μg of antisense ODNs were used per 10 cm dish. To exclude the possibility that the results were due to artefacts after calcium phosphate transfection, control transfections of pGemDHBV and AS ODNs 795 and 874 with the non-liposomal lipid Effectene® (Qiagen, Hilden, Germany) were performed with comparable results. Cells were harvested two days after transfection.

(D) Northern Blot Analysis

Total cellular RNA was prepared using the RNeasy Mini Kit (Qiagen). Samples were treated with 5 U DNase I (GibcoBRL) for 30 min to eliminate plasmid DNA. The RNA was separated on MOPS (morpholinopropanesulfonic acid) buffered formaldehyde containing 1% agarose gels. RNA was blotted onto Nylon membranes (Hybond N, Amersham Pharmacia Biotech, Freiburg, Germany) by capillary transfer using 1.5 M NaCl, 150 mM sodium citrate. RNA was visualized by hybridization with ³²P labeled oligo probes.

(D) RNA Ligation, RT-PCR, Cloning and Sequencing

5 μg of total cellular RNA was ligated by 100 U T4 RNA ligase (New England Biolabs, Schwalbach, Germany) with 40 U RNasin (Promega, Mannheim, Germany) for 1 h at 37° C. RNA was then extracted using the RNeasy kit. During the second step, ligated RNA was reverse transcribed using primer 912− (located downstream from the AS ODN target site, CTGACCATGTCAAAGTCC) and AMV-reverse transcriptase (Superscript II, GibcoBRL) for 90 min at 42° C. The cDNA was amplified by PCR using primers 912− or 860− (located upstream from primer 912−, CAATATTTCTCCTCCTTC) and 2665+ (located downstream from primer 912, AGAGCCTTAGCCAATGTGTAT). 30 cycles (1 min 94° C., 1 min 55° C., 1.5 min 72° C.) were performed. The ligated RNA transcript specificallly generated a 0.4 kbp PCR product which was excised from the agarose gel and cloned. The cloned PCR products were sequenced with ³²P-labeled primers. All enzymatic reactions were performed in the appropriate buffer provided by the manufacturer.

(E) Assessment of Poly(A) Tail Length

Poly(A) tail length was measured as described (Binder et al., EMBO J.13 (1994), 1969-1980). Approx. 5 μg of total cellular RNA was hybridized to 20 pmol AS ODN 2101 (complementary to DHBV nt 2101-2118) and cleaved with RNase H (5 Units, USB, Cleveland, Ohio) in RNase H-Buffer provided by the manufacturer for 30 min at 37° C. This reaction cleaved DHBV mRNAs ˜600 nt upstream of the poly(A) tail. Where indicated, the poly(A) tail was removed by including 50 pmol oligo(dT)₁₂₋₁₈ in the reactions. In another sample, AS ODN 2101 was replaced by AS ODN 2001 complementary to nt 2001-2018. The resulting ˜700 nt fragment served as internal size marker. The reaction products were denatured by urea containing 6% PAGE (Sequagel, National Diagnostics, Atlanta, Ga.), electroblotted on nylon membranes and hybridized with a ³²P labeled probe complementary to nt 2281-2313.

(F) Western Blot Analysis

For immunoblot analysis three different polyclonal antibodies were used: an antibody against DHBV surface antigen (Lambert at al., J.Virol.64 (1990), 1290-1297), an antibody that recognizes all green fluorescent protein variants including fusion proteins (Clontech, Heidelberg, Germany, Cat.#8367-1) and an antibody against transcription factor C/EBP_{α} (Santa Cruz Biotechnology Inc., Santa Cruz, USA, Cat.#sc-61). Cell extracts were denatured, separated by SDS-PAGE (12%) and electroblotted to PVDF membranes (Immobilon, Millipore, Bedford, USA). After blocking, the membrane was incubated with the primary antibody overnight. Using the enhanced chemiluminiscence detection method the sequential steps were carried out according to the instructions of the manufacturer (Pierce, Rockford, USA, Cat. #34080).

(G) Fluorescence Microscopy

For fluorescence microscopy, cells were washed with PBS and fixed for 15 min with 2% paraformaldehyde and 0.05% Triton X in PBS. After washing with PBS, the nuclei were stained with 1 μg/ml propidium iodide and 200 μg/ml RNase A (Qiagen) in PBS. Photos were taken using the same exposure times using specific fluorescence filters for propidium iodide and ECFP.

### Example 2

Generation of Stable 3′ mRNA Fragments by Antisense ODNs Directed Against DHBV preS/S mRNA

Previously AS ODNs as therapy against hepatitis B virus (HBV) in the duck HBV (DHBV) model were evaluated. It was shown that an AS ODN directed against the start of the preS region resulted in a dramatic antiviral effect in primary hepatocytes and in DHBV-infected Pekin ducks. In this example, the molecular mechanisms of AS action in avian and human hepatoma cells cotransfected with a replication-competent DHBV construct and phosphorothioate-modified, 18-mer antisense ODNs, was further investigated. The DHBV genome consists of a partially double-stranded, circular DNA molecule of 3021 bp length (Sprengel and Will (1988), Duck hepatitis B virus. In Virus Diseases in Laboratory and Captive Animals, G. Darai, ed. (Boston, Mass.) Nijhoff Publishers pp.363-386). The pregenomic (pg) mRNA (nt 2530-3021/1-2796) which codes for the viral reverse transcriptase/DNA polymerase and the viral core proteins and the subgenomic (sg) mRNA (nt 740-2796) which codes for the surface (preS/S and S) proteins are two major transcripts of the DHBV genome. As illustrated in FIG. 1a the AS ODN DHBV795 targets simultaneously the first AUG on the sg mRNA and an internal AUG of the pg mRNA. Northern blot analysis of cotransfected avian hepatoma cells revealed that the target mRNAs were not completely degraded. Instead a distinct band slightly smaller than sg mRNA was specifically generated by AS treatment (FIG. 1b). This band was constantly detected over a period of 4 days after transfection. Hybridization of the Northern blot with the probes depicted in FIG. 1a indicated that the AS ODN mediated cleavage of both target mRNAs with subsequent degradation of the 5′ but not the 3′ fragment. Complete degradation of the 5′ fragment was confirmed by primer extension analysis. A similar 3′ cleavage fragment was observed after cotransfection with AS ODN DHBV795 in human hepatoma cells as shown in FIG. 1c. Furthermore, transfections with an AS ODN directed against the third AUG (AS DHBV874) resulted in a slightly smaller 3′ cleavage fragment (FIG. 1c, lane 4).

### Example 3

Characterization of the Termini of the 3′ mRNA Fragment

The stability and degradation of mRNAs are commonly thought to be determined by their 5′ cap structures and 3′ poly(A) tails. Therefore, the termini of the 3′ cleavage fragments were investigated in more detail. The existence of a 5′ cap structure can be experimentally examined by immunoprecipitation. This technique was applied using the cap specific antibody H20 (Bochnig et al., Eur.J.Biochem.168 (1987), 461-467) in pilot experiments, hypothesizing that capped uncleaved DHBV mRNA would be immunoprecipitated whereas uncapped cleaved fragments would not. Unfortunately, sufficient quantities of capped mRNA species from total cellular RNA preparations (either DHBV or glyceraldehyde-3-phosphate dehydrogenase (GAPDH) as control) could not be precipitated for Northern hybridization. As the antibody H20 is directed against virtually all capped cellular messenger and small nuclear RNAs, this technique was not useful because unconcentrated total cellular RNA preparations had to be used.

If some or all 3′ cleavage fragments were uncapped they should be efficiently ligated using T4 RNA ligase. This reaction requires a monophosphorylated 5′ terminus and is prevented by the cap structure. Hence, efficient ligation should only occur in uncapped RNAs. To address this question, a strategy illustrated in FIG. 2a was performed (modified from Couttet et al., PNAS USA 94 (1997), 5628-5633). First, the mRNA was circularized using T4 RNA ligase. In the next step cDNA synthesis across the ligated termini was made using primer 912 situated upstream to the cleavage sites and avian myeloblastosis virus reverse transcriptase. This copy of the poly(A) tail and its surrounding sequences were amplified in the third step by using Taq DNA polymerase and two primers that hybridize on both sides of the junction region. Indeed, an expected 0.4 kbp PCR product was detected (FIG. 2b, lane 4). Sequence analysis of this PCR product revealed that the 3′ cleavage fragments were successfully ligated (FIG. 2c). In contrast, uncleaved sg DHBV mRNA was not efficiently ligated and amplified by this procedure. Note that the PCR primers used here also yielded 1.3 and 0.8 kbp fragments that were transcribed from the pg mRNA and/or the DHBV expression plasmid and did not require the ligation step (FIG. 2b lanes 1,2,7). In keeping with this notion, a control plasmid (pDHBVsg) encoding only the sg mRNA did not generate any PCR products (FIG. 2b, lane 3).

Sequence analysis of the PCR products revealed furthermore that the 5′ ends of the cleavage fragments mapped to the 3′ region of the AS ODN/mRNA heteroduplex while the 3′ end was intact. The cleavage sites of 18 clones were analyzed and are illustrated in FIG. 2d. This cleavage pattern showed that the majority of these events occurred in the distal 3′ region on the mRNA in the heteroduplex. The 5′-most cleavage site on the mRNA was 11 nucleotides from the 3′ end of the ODN. The 3′-most cleavage site extended 3 residues past the heteroduplex into the single strand 3′ overhang region. No cleavage sites could be observed in the 5′ region on the mRNA in the heteroduplex and upstream of the heteroduplex in the single strand 5′ overhang region.

To assess the poly(A) tail length, total cellular RNA from DHBV control (FIG. 3, lanes 1-4) and AS ODN treated cells (FIG. 3, lanes 5-8) were hybridized to AS ODNs complementary to the DHBV 3′ UTR and further cleaved in vitro using RNase H as described (Binder et al., 1994). Two different AS ODNs were used to obtain an internal control of fragment size. Addition of oligo(dT)₁₂₋₁₈ primer to the reactions lead to 3′ UTR RNA fragments of 700 and 600 nt, respectively, that lacked a poly(A) tail (FIG. 3, lanes 2, 3, 6 and 7), whereas differences of fragment size after omission of oligo(dT)₁₂₋₁₈ directly reflected poly(A) tail length (FIG. 3, lanes 4 and 8). The observed poly(A) tail length distribution did not differ significantly in both populations comprising a peak at approx. 100-200 adenosines with a discrete smear of degradation products.

### Example 4

The 3′ mRNA Fragments are Efficiently Translated into N-Truncated Proteins

Next it was analyzed whether the stable 3′ mRNA cleavage fragments could still be translated into protein. Western blot analyses of viral preS/S proteins isolated from cotransfected avian and human hepatoma cells were performed (FIG. 4). The pattern of viral preS/S proteins in control transfected hepatoma cells without AS ODNs was similar to that obtained during natural infection. The first AUG (nt 801) is predominantly used yielding gp36. Low level translation initiating from the internal AUGs is observed, most likely due to leaky scanning. Initiation of translation at the second AUG (nt 825) leads to gp35, initiation at the third AUG (nt 882) to gp 33 and initiation at the fourth AUG (nt 957) to gp 30. When avian (LMH) or human hepatoma cells (HuH-7 and HepG2) were cotransfected with AS ODN DHBV795, translation of viral preS/S proteins was not abolished. However, instead of the predominant gp36, preS/S proteins with a smaller apparent size than wild type were detected. The two smaller proteins could be identified as gp35 translated from the next internal AUG located 24 nt downstream of the first AUG and as gp33 translated from the third AUG located 81 nt downstream of the first AUG. This was confirmed by site directed mutagenesis of the respective start codons as well as probing the Western blot with an antibody that recognized aa 9-28 (corresponding nt 825-884) of the preS/S protein. Furthermore, AS ODNs directed against different regions of the viral preS/S mRNA ORF (e.g., second AUG, third AUG) always resulted in translation of N-truncated preS/S proteins starting at the AUG located downstream from the AS target site as shown in FIG. 4b for AS ODN DHBV874. This AS ODN is directed against the third AUG and caused a shift of translation initiation to the fourth AUG yielding gp30. The residual expression of gp36 in this experiment may be due to incomplete cleavage by the AS ODN DHBV874 in HuH-7 cells (see FIG. 1c for the corresponding Northern blot).

### Example 5

AS ODN-Mediated Shift of Translation Initiation Results in Relocation of Fluorescence of ECFP-Golgi and Shift of Isoform Expression of C/EBP_{α}

To evaluate whether the generation and translation of stable 3′ cleavage fragments would apply to other, non viral mRNA targets, AS ODNs were designed for the ECFP-Golgi (enhanced cyan fluorescent protein) and C/EBP_{α} mRNAs. ECFP-Golgi is a fusion protein consisting of enhanced cyan fluorescent protein (AUG of ECFP: nt 864-866) and a sequence encoding a region of human beta 1.4 galactosyltransferase which contains the membrane anchoring signal peptide that directs the fusion protein to the Golgi apparatus (AUG of the fusion protein: nt 597-599; Llopis et al., PNAS USA 95 (1998), 6803-6808). It was hypothesized that an AS ODN-mediated shift of translation initiation to the downstream ECFP-AUG should result in a protein lacking the membrane anchoring protein. Therefore its fluorescence should be diffusely located throughout the cell. Indeed, AS ODN ECFP-Golgi689 rearranged the intracellular distribution of the fluorescence protein accordingly (FIG. 5a). Northern blot analysis revealed the generation of stable 3′ cleavage fragments and the degradation of the corresponding 5′ fragments (FIG. 5b). In cotransfection experiments using a different AS ODN (ECFP-Golgi761) binding 72 nt 3′ from AS ODN 689 a slightly smaller 3′ cleavage fragment was generated (FIG. 5b). Western blot analysis demonstrated the expression of a novel truncated protein (FIG. 5c).

As a third example, the mammalian transcription factor C/EBP_{α} was examined as an AS ODN target for the following reasons. Various isoforms of the transcription factor C/EBP_{α} are known that arise from unique C/EBP_{α} mRNAs by differential initiation of translation. Full length C/EBP_{α} (p42) inhibits cell proliferation, a smaller isoform p30 was shown to be a transcriptional activator lacking the antimitotic activity. It was investigated whether AS ODN could shift isoform expression towards the p30 isoform. Avian hepatoma cells were cotransfected with a plasmid encoding C/EBP_{α} and AS ODN C/EBP_{α}516. FIG. 6 shows that this AS ODN directed against the methionine of p42 shifted the C/EBP_{α} isoform ratio to the isoform p30.

The foregoing is meant to illustrate, but not to limit, the scope of the invention. The person skilled in the art can readily envision and produce further embodiments, based on the above teachings, without undue experimentation.

What is claimed is:

1. A method for the generation of a 3′ RNA cleavage fragment from a target RNA, said method comprising the following steps:
(a) transfecting a cell containing a target RNA or incubating an extract obtained from said cell with an antisense oligonucleotide capable of hybridizing to said target RNA or an expression vector containing a DNA molecule which is operatively linked to regulatory elements allowing transcription of said antisense oligonucleotide in said cell; and
(b) maintaining or culturing said cell or incubating said extract for a sufficient period of time and under conditions such that said target RNA is degraded and a stable 3′ RNA cleavage fragment produced.

2. The method of claim 1, said method further comprising the following step:
(c) detecting said 3′ RNA cleavage fragment in said cell or said extract.

3. The method of claim 1, wherein said RNA is an mRNA.

4. The method of claim 1, wherein said antisense oligonucleotide has a length of at least 8 nucleotides.

5. The method of claim 1, wherein said antisense oligonucleotide is phosphorothioate-modified.

6. The method of claim 1, wherein said cell is a mammalian cell, a bacterial cell, an insect cell or a yeast cell.

7. A method for the preparation of an N-terminally truncated polypeptid, said method comprising the following steps:
(a) transfecting a cell containing a target mRNA or incubating an extract obtained from said cell with an antisense oligonucleotide capable of hybridizing to said target mRNA or an expression vector containing a DNA molecule which is operatively linked to regulatory elements allowing transcription of said antisense oligonucleotide in said cell;
(b) maintaining or culturing said cell or incubating said extract for a sufficient period of time and under conditions such that said target mRNA is degraded and a polypeptid translated from the 3′ mRNA cleavage product; and, optionally,
(c) detecting and functionally characterizing an N-terminally truncated polypeptid translated from a 3′ mRNA cleavage product as a result of steps (a) and (b).

8. The method of claim 7, wherein said antisense oligonucleotide has a length of at least 8 nucleotides.

9. The method of claim 7, wherein said antisense oligonucleotide is phosphorothioate-modified.

10. The method of claim 7, wherein said cell is a mammalian cell, a bacterial cell, an insect cell or a yeast cell.

11. Use of the method of claim 1 for studying the stability and translational efficiency of uncapped mRNAs.

12. Use of the method of claim 1 for generating novel polypeptides with desired properties from an endogenous cellular repertoire.

13. Use of the method of claim 1 for evaluating possible side effects of an antisense therapy.

14. Use of the method of claim 1 for generating non-translated RNA fragments which may serve as decoy for interfering with cellular functions or the life cycle of viruses or microorganisms.
